(19) United States
(12) Patent Application Publication
(10) Pub. No.: US 2014/0275870 A1
(43) Pub. Date: Sep. 18, 2014

(54) CONTINUOUS NONINVASIVE MEASUREMENT OF ANALYTE CONCENTRATION USING AN OPTICAL BRIDGE

(71) Applicants: Hannu HARJUNMAA, Holden, MA (US); Rebecca Burrell, Rowley, MA (US); Stevan Kun, Worcester, MA (US)

(72) Inventors: Hannu HARJUNMAA, Holden, MA (US); Rebecca Burrell, Rowley, MA (US); Stevan Kun, Worcester, MA (US)

(73) Assignee: Grove Instruments Inc., Worcester, MA (US)

(21) Appl. No.: 13/835,143

(22) Filed: Mar. 15, 2013

(51) Int. Cl. A61B 5/1455; A61B 5/1495; A61B 5/00; A61B 5/145; A61B 5/0205

(52) U.S. Cl. CPC A61B 5/1455; A61B 5/14532; A61B 5/0205; A61B 5/0004; A61B 5/1495; A61B 5/02416
USPC 600/316; 600/310; 600/322

(57) ABSTRACT

A method may include directing a radiation beam at a sample, the beam including two periods of radiation having different wavelengths, an analyte in a fluid within the sample having different absorption coefficients for the two different wavelengths, detecting the beam with a detector when the sample is in a first fluid state, the detector configured to generate an output signal proportional to an intensity of the beam at each of the two different wavelengths, detecting the beam with the detector when the sample is in a second fluid state, the sample transitioning from the first fluid state to the second fluid state by a pulsation of the sample, obtaining estimates of an amount of fluid at the first and second fluid states, and determining an analyte concentration estimate based on the output signal and the estimate of the amount of fluid at the first and second fluid states.

### FIELD OF THE DISCLOSURE

The present disclosure generally relates to the field of biomedical testing. More specifically, the present disclosure relates to methods and apparatus for noninvasive measurement of concentration of analytes in body tissues.

### BACKGROUND OF THE DISCLOSURE

Noninvasive diagnosis and measurement of blood glucose concentration has attracted tremendous attention in the past two decades because of the emergence of diabetes as an epidemic, particularly when associated with an increased overall obesity of the population. Noninvasive measurement of glucose offers the potential for increased frequency of testing, and thus, enable tighter control of blood glucose concentrations through concomitant adjustment of insulin doses. Noninvasive detection techniques also offer the potential for a portable, closed-loop system for monitoring and regulating insulin dosage. These prospective advantages have led to considerable interest in the commercialization of noninvasive glucose monitoring devices.

Many existing portable end-user devices for measuring blood glucose require puncturing the fingertip to obtain a blood sample. The blood sample is then placed on a test strip that indicates the glucose concentration. These devices are compact and reasonably accurate, but puncturing the fingertip to obtain a blood sample is inconvenient, painful, and poses a risk of infection.

A number of attempts have been made to measure blood glucose concentration noninvasively by measuring tissue absorption of light radiation in the near infrared energy spectrum-approximately 650 nm to 2700 nm. U.S. Pat. No. 5,099,123 to Harjunmaa et al., which is incorporated herein by reference in its entirety, discloses a balanced differential (or Optical Bridge) method for measurement of analyte concentration in turbid matrices, i.e., body fluids and tissue. The method utilizes two wavelengths: (1) a principal wavelength, which is highly absorbed in the target analyte, and (2) a reference wavelength, selected using a balancing process, which is not (or much less) absorbed in the target analyte. The two wavelengths are selected to have substantially identical extinction coefficients in the background matrix. When a radiation beam comprising the two wavelengths in alternate succession is applied to the sample tissue matrix, an alternating signal synchronous with the wavelength alternation is registered in a signal detector measuring the radiation transmitted or backscattered by the matrix. The amplitude of the alternating signal is proportional to the concentration of the target analyte in the sample matrix. During the measurement, the Optical Bridge balancing process is used to vary the two alternating wavelengths and their relative intensities such that in the absence of analyte, the detector signal is essentially zero. That is, the Optical Bridge uses the two near infra-red wavelengths to “null out” the background absorption so that the analyte concentration becomes much more visible.

U.S. Pat. No. 5,178,142 to Harjunmaa et al., which is incorporated herein by reference in its entirety, discloses a method of changing the extracellular to intracellular fluid ratio of the tissue matrix by varying the mechanical pressure on the tissue, and zeroing the transmitted/reflected signal (balancing) when there is a minimum level of analyte present in the sample.

U.S. Pat. No. 7,003,337 to Harjunmaa et al., which is incorporated herein by reference in its entirety, discloses continuous estimation of the amount of fluid containing the target analyte within the sample using another radiation (such as green light which is absorbed by hemoglobin), and combining the output of the sample detector with the fluid volume estimate to calculate the analyte concentration. Moreover, U.S. Pat. No. 8,175,666 to Harjunmaa et al., which is incorporated herein by reference in its entirety, discloses a method of producing a radiation beam using three fixed-wavelength laser diodes instead of tuning the laser wavelengths during use.

Other related patents include U.S. Pat. Nos. 5,112,124; 5,137,023; 5,183,042; 5,277,181 and 5,372,135, each of which is incorporated by reference herein in its entirety.

### SUMMARY OF THE DISCLOSURE

In accordance with an embodiment, a method for measuring an analyte in a fluid within a sample with an optical system may include directing a radiation beam at the sample, the beam including at least two periods of radiation having different wavelengths, the analyte having different absorption coefficients for the two different wavelengths. The method may further include detecting the beam with a detector when the sample is in a first sample fluid state having a first amount of fluid, wherein the detector is configured to generate an output signal proportional to an intensity of the beam at each of the two different wavelengths, obtaining an estimate of the first amount of fluid, detecting the beam with the detector when the sample is in a second sample fluid state having a second amount of fluid, wherein the sample transitions from the first sample fluid state to the second sample fluid state by a pulsation of the sample, obtaining an estimate of the second amount of fluid, and determining an estimate of a concentration of the analyte in the fluid based on the output signal, the estimate of the first amount of fluid, and the estimate of the second amount of fluid.

In accordance with another embodiment, a method for measuring an analyte in blood within tissue of a subject may include directing a radiation beam at the tissue, the beam including at least two periods of radiation having different wavelengths, the analyte having different absorption coefficients for the two different wavelengths. The method may further include detecting the beam with a detector when the tissue is in a first blood state having a first amount of blood, wherein the detector is configured to generate an output signal proportional to an intensity of the beam at each of the two different wavelengths, obtaining an estimate of the first amount of blood, detecting the beam with the detector when the tissue is in a second blood state having a second amount of blood, wherein the tissue transitions from the first blood state to the second blood state by a pulse from a heartbeat of the subject, obtaining an estimate of the second amount of blood, and determining an estimate of a concentration of the analyte in the blood based on the output signal, the estimate of the first amount of blood, and the estimate of the second amount of blood.

In accordance with yet another embodiment, a method for measuring an analyte in a fluid within a sample may include directing a radiation beam at the sample, the beam including at least two periods of radiation having different wavelengths, the analyte having different absorption coefficients for the two different wavelengths. The method may further include detecting the beam with a detector when the sample is in a first sample fluid state having a first amount of fluid, wherein the detector is configured to generate an output signal proportional to an intensity of the beam at each of the two different wavelengths, obtaining an estimate of the first amount of fluid, detecting the beam with the detector when the sample is in a second sample fluid state having a second amount of fluid, wherein the sample transitions from the first sample fluid state to the second sample fluid state by pulsations of the sample, obtaining an estimate of the second amount of fluid, and determining estimates of a concentration of the analyte in the fluid for a predetermined number of pulsations based on the output signals, the estimate of the first amount of fluid, and the estimate of the second amount of fluid.

Reference will now be made in detail to exemplary embodiments of the present disclosure described above and illustrated in the accompanying drawings. Wherever possible, the same reference numbers will be used throughout the drawings to refer to the same or like parts.

In this respect, before explaining multiple embodiments of the present disclosure in detail, it is to be understood that the present disclosure is not limited in its application to the details of construction and to the arrangements of the components set forth in the following description or illustrated in the drawings. The present disclosure is capable of embodiments in addition to those described and of being practiced and carried out in various ways. Also, it is to be understood that the phraseology and terminology employed herein, as well as the abstract, are for the purpose of description and should not be regarded as limiting.

The accompanying drawings illustrate certain exemplary embodiments of the present disclosure, and together with the description, serve to explain the principles of the present disclosure. As such, those skilled in the art will appreciate that the conception upon which this disclosure is based may readily be used as a basis for designing other structures, methods, and systems for carrying out the several purposes of the present disclosure. It is important, therefore, to recognize that the claims should be regarded as including such equivalent constructions insofar as they do not depart from the spirit and scope of the present disclosure.

### BRIEF DESCRIPTION OF THE DRAWINGS

FIG. 1 illustrates a schematic diagram of an optical system, according to an exemplary disclosed embodiment; and

FIG. 2 illustrates a block diagram for a process of determining a target analyte concentration, according to an exemplary disclosed embodiment.

### DETAILED DESCRIPTION

In an exemplary embodiment, an optical system of the present disclosure may be configured to measure the concentration of a target analyte in a fluid within a sample matrix. The sample matrix may include three components: non-fluid, bound fluid, and unbound fluid. The non-fluid and bound fluid components may be generally fixed, and together may be referred to as the background matrix. In human tissue, for example, the background matrix may include the cellular matrix and intra-cellular fluid. The unbound fluid may generally not be fixed in the sample matrix, and may freely circulate through the fixed background matrix of the sample matrix. In human tissue, for example, the unbound fluid may include blood and other substances dissolved in or otherwise contained within the blood. An inrush of the unbound fluid to the sample matrix may be caused by, for example, natural pulsations due to beating of the heart.

To measure and analyze the target analyte concentration, the optical system may utilize “Optical Bridge” technology. As used herein, the term “Optical Bridge” may refer to an apparatus and/or method for quasi-simultaneous differential optical measurement of very small absorbance differences of a sample, performed at one or more wavelength pairs.

FIG. 1 illustrates a schematic diagram of an optical system 1, according to an exemplary disclosed embodiment. System 1 may be configured to noninvasively measure the concentration of a target analyte (e.g., glucose) in a fluid (e.g., blood) within a sample matrix (e.g., a tissue matrix). System 1 may include a radiation source 10 configured to produce a beam of electromagnetic radiation. This beam may include time multiplexed components (principal, reference, and blood amount) of desired wavelengths. That is, source 10 may be configured to produce a beam of electromagnetic radiation that alternates at a particular frequency between a “principal” wavelength (λ_{P}), a “reference” wavelength (λ_{R}), and a “blood” wavelength. In one embodiment, principal wavelength λ_{P} may be more strongly absorbed by a target analyte, such as glucose, than reference wavelength λ_{R}. For example, principal wavelength λ_{P} may be between approximately 1550 and 1700 nm, and reference wavelength λ_{R} may be between approximately 1350 and 1430 nm. The blood wavelength may be close to 525 nm (isosbestic point for hemoglobin).

In certain embodiments, source 10 may include one or more light sources, such as laser diodes, configured to emit a probe beam 202. The laser diodes may be controlled by a suitable laser control unit 21 and laser driver 20 as known in the art.

System 1 may also include a microcontroller 104 and a memory 106. Microcontroller 104 may be configured to generate signals for controlling various components of system 1, including principal and reference wavelengths λ_{P} and λ_{R}, principal and reference wavelength intensities I_{P} and I_{R}, and green light intensity of probe beam 202. Memory 106 may be configured to store various parameters and measurement results of system 1, and may be in communication with microcontroller 104.

Although not illustrated, it should be appreciated that system 1 may include any suitable power source, such as a battery, to power the components of system 1. In certain embodiments, system 1 may also include a force transducer 30 and an accelerometer 31, each in communication with microcontroller 104. Force transducer 30 may include any suitable transducer configured to convert a mechanical energy signal to an electrical energy signal. Accelerometer 31 may include any suitable accelerometer configured to measure an acceleration associated with system 1. Microcontroller 104 may be configured to receive data from force transducer 30 and accelerometer 31 and manage the accuracy and power spent by system 1 based on data from one or both of force transducer 30 and accelerometer 31. More specifically, force transducer 30 and/or accelerometer 31 may detect movements or any other disturbances to system 1 that may lead to less accurate measurements by system 1. Force transducer 30 and/or accelerometer 31 may signal force and/or acceleration measurements to microcontroller 104, and if such measurements exceed a specified threshold, the microcontroller 104 will not commence measurements until the force and/or acceleration measurements retreat below the threshold. Accordingly, system 1 may be prevented from wasting power on measurements that may likely not be accurate.

A sample specimen 80, such as, for example, an earlobe, finger, lip, cheek, nasal septum, tongue, or the skin between the fingers or toes of the subject, may be positioned between a diffuser plate 70 and a sample detector lens 92 of system 1. Positioning diffuser plate 70 upstream of the path of probe beam 202 relative to sample specimen 80 may minimize the effects of the variation in the scattering properties of sample specimen 80. In certain embodiments, diffuser plate 70 may be an integral part of the laser package, and sample detector lens 92 may be an integral part of the detector package. Moreover, in some embodiments, system 1 may include an appropriate holding mechanism 50, such as a clamp, configured to hold together and/or reposition source 10 and sample detector 91.

Probe beam 202 may be transmitted through sample specimen 80, and may be focused by sample detector lens 92. Probe beam 202 may then be transmitted to a sample detector 91. Sample detector 91 may be configured to detect the intensity at each of the periods of principal and reference wavelengths λ_{P} and λ_{R}, as well as the green light intensity, and may send an electrical signal 302 to an amplifier 26. An analog-to-digital converter 60 may be in communication with amplifier 26 and microcontroller 104. Analog-to-digital converter 60 may include any suitable analog-to-digital converter configured to convert the continuous-intensity and continuous-time signal associated with electrical signal 302 to a discrete-intensity and discrete-time signal. The converted signal may then be sent to microcontroller 104 as an output signal 308. Output signal 308 contains three time-multiplexed components: a green light signal, a reference signal, and a principal signal. Within microcontroller 104, output signal 308 may be digitally converted to the Optical Bridge signal that is proportional to the difference (or ratio) of the principal and reference intensities detected by sample detector 91.

According to an embodiment, three different types of probe beam attenuations may be distinguished by system 1. The first type may be the background matrix, the second type may be the target analyte, and the third type may be the unbound fluid attenuation.

The background matrix attenuation may result from the absorption of probe beam 202 by sample constituents whose concentrations may be substantially constant throughout fixed sample compartments. The target analyte attenuation may be caused by absorption of probe beam 202 by the target analyte (e.g., glucose), which may be mostly concentrated in the unbound fluid (e.g., blood). The unbound fluid, along with the target analyte, may be substantially displaced from sample specimen 80 in between pulses of the heart, and an inrush of the unbound fluid, along with the target analyte, to sample specimen 80 may be caused by the pulses of the heart. Since the target analyte concentration in the unbound fluid may be different than the target analyte concentration in the background matrix (e.g., intracellular concentration), the average target analyte concentration in the beam path may change as a result of the natural pulsations of the heart. This concentration change may allow the target analyte to be detected by system 1.

Principal wavelength λ_{P} of probe beam 202 may be set to have high attenuation by the target analyte. Principal wavelength intensity I_{P} may be set to achieve an optimal transmitted signal intensity. Reference wavelength λ_{R} of probe beam 202 may be either pre-set or selected during an Optical Bridge balancing process. Reference wavelength intensity I_{R} may be adjusted before each measurement as explained below.

In the Optical Bridge balancing process, microcontroller 104 may adjust reference wavelength λ_{R} to have substantially the same absorption in low-blood content tissue as principal wavelength λ_{P}. Probe beam 202 may be directed at sample section 80, and the Optical Bridge may be balanced or nulled by adjusting reference wavelength λ_{R} and its intensity I_{R} to obtain a substantially-zero Optical Bridge signal when the unbound fluid is displaced by the natural pulsation cycle of the heart (i.e., in between heart beats). In other embodiments, reference wavelength intensity I_{R} may be set by microcontroller 104, while principal wavelength intensity I_{P} may be adjusted by microcontroller 104 to balance the Optical Bridge. Accordingly, by the Optical Bridge balancing process, the light intensities and reference wavelength λ_{R} may be adjusted such that the baseline absorption (indicated by Optical Bridge signal) may be essentially zero when there is minimal fluid and analyte in the optical path, and the differential absorption between principal wavelength λ_{P} and reference wavelength λ_{R} may be minimal.

Upon completion of the Optical Bridge balancing process, continuous measurement of the target analyte may commence. As alluded to above, system 1 may include Optical Bridge technology that exploits the principle that sample section 80 may include a relatively higher proportion of fluid with the target analyte due to pulses from heartbeats than in between pulses from heartbeats. That is, an inrush of the unbound fluid to sample section 80 may be caused by pulses of the heart. Accordingly, the natural pulsations of the heart may cause sample section 80 to absorb probe beam 202 differently at principal wavelength λ_{P} and reference wavelength λ_{R}. This change in absorption of principal wavelength λ_{P} and reference wavelength λ_{R} may result in a non-zero Optical Bridge signal. The measurements may continue until a predetermined sample period is reached. For example, the predetermined period may be time-dependent or pulse-dependent. System 1 may continuously take measurements for a predetermined amount of time, such as, for example, 10 seconds, or may continuously take measurements for a predetermined amount of pulses, such as, for example, 100.

Microcontroller 104 of system 1 may then process the obtained measurements to determine the target analyte concentration. FIG. 2 illustrates a block diagram for a process of determining the target analyte concentration, according to an exemplary disclosed embodiment.

At step 502, microcontroller 104 may first determine whether conditions are suitable for measurements. Particularly, microcontroller 104 may receive data from one or both of force transducer 30 and accelerometer 31 to determine force and/or acceleration measurements exceed the specified threshold due to the subject's activity. If the measurements do not, the microcontroller may then begin obtaining measurements.

Upon obtaining the measurements at step 503, microcontroller 104 may receive output signal 308 and green light signals and perform an unbound fluid amount calculation based on green light signals at step 507. Optical Bridge signal may be represented by the following equation:

OBS=CCS·UFA·TAC+CCI (Eq. 1)

where:

OBS=Optical Bridge signal;

CCS=calibration constant slope;

UFA=unbound fluid amount;

TAC=target analyte concentration;

CCI=calibration constant intercept; and

TAA=target analyte amount=UFA·TAC.

When the Optical Bridge has been balanced for a measurement, the magnitude of the measured Optical Bridge signal OBS may represent the difference in the absorbed light intensity at principal wavelength λ_{P} and reference wavelength λ_{R} resulting from the absorption of the target analyte within sample section 80. This difference may be proportional to the difference of the absorption by the target analyte at principal wavelength λ_{P} and reference wavelength λ_{R}, as well as to the amount of the target analyte TAA in sample section 80. The amount of target analyte TAA in the unbound fluid of the sample may be calculated as the product of the unbound fluid amount UFA and the target analyte concentration TAC in the unbound fluid. The difference of the absorption by the target analyte at principal wavelength λ_{P} and reference wavelength λ_{R} may be determined based on calibration constant slope CCS and calibration constant intercept CCI. Calibration constant slope CCS and calibration constant intercept CCI may be determined by a calibration process described in greater detail below. In order to obtain the concentration of the target analyte TAC (e.g., glucose concentration) in the unbound fluid (e.g., blood), the Optical Bridge signal OBS may be normalized with the amount of the unbound fluid UFA. Since the signals may be derived from a change in blood content, the glucose estimate pertains specifically to blood glucose and is not affected by the interstitial glucose.

Principal and reference wavelength intensity signals may be dependent on the variation of the total amount of fluid in the optical path. This dependence may be non-linear and relative. Similarly, a blood signal may be dependent on the variation of the amount of unbound fluid UFA in the optical path, and this dependence may also be non-linear and relative. Accordingly, microcontroller 104 may perform mathematical modeling and self-normalization using the blood signal and the principal and reference wavelength intensity signals to calculate estimates of the unbound fluid amount UFA at step 507. The estimates of the unbound fluid amount UFA may include an estimate of a fluid amount at a first fluid state in between heart pulses and an estimate of a fluid amount at a second fluid state when an inrush of the unbound fluid enters sample section 80 due to heart pulses.

Multiple measurements may be obtained during and after each pulse. These measurements may be processed at step 509. Microcontroller 104 may perform a regression of the Optical Bridge measurements OBS versus the calculated unbound fluid amounts UFA over a period beginning before each pulse and ending after each pulse. The slope of the regression line may be directly correlated to the target analyte concentration TAC. This slope may be independent of the amount of unbound fluid entering sample section 80, and may also be independent of the speed into which the unbound fluid enters sample section 80.

As alluded to above, the measurement process may be calibrated prior to performing predictive estimations of the target analyte concentration TAC. The calibration constants CCS and CCI may be determined at step 521 by a calibration algorithm. Such a calibration algorithm may perform the above-described measurement process, except that during the calibration process, the concentration of the target analyte may be a known quantity. A suitable number of measurement sequences may be performed on samples with varying and known concentrations of the target analyte. The measurement sequence for calibration may be identical to the one used in predictive estimation, except that at the end of the procedure, the calibration constant(s), rather than the analyte concentration, may be calculated and stored in memory 106 for future reference by microcontroller 104. Using the relationship of Eq. 1, the calibration algorithm may calculate the calibration constant(s) by performing a regression between the known concentrations of the target analyte and the other calculated parameters of Eq. 1, and may determine the calibration constants from the regression. Generally, there may be one multiplication calibration constant CCS and another additive constant CCI. These calibration constants may be later used to calculate the target analyte concentration TAC in a predictive measurement where the analyte concentration is unknown at step 517.

At step 517, microcontroller 104 may calculate an estimate of the target analyte concentration TAC in the unbound fluid using the measured system parameters and the above-described parameters calculated at steps 507 and 509, in combination with the calibration coefficients CCS and CCI determined at step 521. The target analyte concentration TAC may be calculated based upon the relationship described in Eq. 1. The estimate of the target analyte concentration TAC may be calculated for each pulse, and the average target analyte concentration TAC estimate may be calculated for the total number of pulses for the measurement period. The average target analyte concentration TAC estimate may then be displayed, digitally or otherwise, at step 522.

System 1 and its corresponding methods of measurement may provide a number of features. For example, measurements on sample section 80 may be done based on the natural pulsatile blood volume variation caused by the subject's heartbeats and without exerting external pressure to sample section 80. As such, the use of a compression mechanism to displace blood from sample section 80 may be obviated, simplifying the construction of system 1 and minimizing the costs of system 1. In addition, basing the measurements on the natural pulsation of the subject's heartbeats may eliminate measurement inaccuracies caused by existing compression mechanisms. For example, inaccuracies due to tissue rebound and air gaps in sample section 80 produced by the compression mechanism may be avoided.

It should also be appreciated that system 1 may be a standalone system or can operate in conjunction with a mobile or stationary device to facilitate display or storage of data, and to signal healthcare personnel when therapeutic action is needed, if the device is used for continuous monitoring of a diagnostic parameter associated with a disease state. Mobile devices may include, but are not limited to, handheld devices and wireless devices distant from, and in communication with, system 1. Stationary devices may include, but are not limited to, desktop computers, printers and other peripherals that display or store the results of the test. In an exemplary embodiment, results from microcontroller 104 may be transferred directly to an external mobile or stationary device to facilitate display or storage of data. For example, the results from microcontroller 104 may be displayed or stored on a remote processing device 29, such as a PC using a PC interface, such as an USB port, IRDA port, BLUETOOTH® or other wireless link. In yet another embodiment, remote processing device 29 may include a printer, and the results may be transmitted wirelessly or via a cable to the printer, and the printer may print the results to be used by attending medical personnel.

Any aspect set forth in any embodiment may be used with any other embodiment set forth herein. The many features and advantages of the present disclosure are apparent from the detailed specification, and thus, it is intended by the appended claims to cover all such features and advantages of the present disclosure which fall within the true spirit and scope of the present disclosure. Further, since numerous modifications and variations will readily occur to those skilled in the art, it is not desired to limit the present disclosure to the exact construction and operation illustrated and described, and accordingly, all suitable modifications and equivalents may be resorted to, falling within the scope of the present disclosure.

What is claimed is:

1. A method for measuring an analyte in a fluid within a sample with an optical system, comprising:
directing a radiation beam at the sample, the beam including at least two periods of radiation having different wavelengths, the analyte having different absorption coefficients for the two different wavelengths;
detecting the beam with a detector when the sample is in a first sample fluid state having a first amount of fluid, wherein the detector is configured to generate an output signal proportional to an intensity of the beam at each of the two different wavelengths;
obtaining an estimate of the first amount of fluid;
detecting the beam with the detector when the sample is in a second sample fluid state having a second amount of fluid, wherein the sample transitions from the first sample fluid state to the second sample fluid state by a pulsation of the sample;
obtaining an estimate of the second amount of fluid; and
determining an estimate of a concentration of the analyte in the fluid based on the output signal, the estimate of the first amount of fluid, and the estimate of the second amount of fluid.

2. The method of claim 1, wherein the sample is tissue, the fluid is blood, and the analyte is glucose.

3. The method of claim 2, wherein the pulsation is a pulse from a heartbeat.

4. The method of claim 3, further comprising determining estimates of the concentration of the analyte in the fluid for a number of heartbeats.

5. The method of claim 3, further comprising determining estimates of the concentration of the analyte in the fluid for a period of time.

6. The method of claim 1, wherein the second amount of fluid is larger than the first amount of fluid.

7. The method of claim 1, wherein, when the sample transitions from the first sample fluid state to the second sample fluid state, a volume of fluid increases in the sample.

8. The method of claim 1, wherein the sample transitions from the first sample fluid state to the second sample fluid state without external pressure being applied to the sample.

9. The method of claim 1, further comprising determining estimates of the concentration of the analyte in the fluid for a number of pulsations, and averaging the estimates of the concentration of the analyte in the fluid.

10. The method of claim 1, wherein the optical system includes an accelerometer, and further comprising managing the power spent by the optical system based on data from the accelerometer.

11. The method of claim 1, wherein the optical system includes a force transducer, and further comprising managing the power spent by the optical system based on data from the force transducer.

12. The method of claim 1, further comprising transmitting data from the optical system to a remote processing device.

13. A method for measuring an analyte in blood within tissue of a subject, comprising:
directing a radiation beam at the tissue, the beam including at least two periods of radiation having different wavelengths, the analyte having different absorption coefficients for the two different wavelengths;
detecting the beam with a detector when the tissue is in a first blood state having a first amount of blood, wherein the detector is configured to generate an output signal proportional to an intensity of the beam at each of the two different wavelengths;
obtaining an estimate of the first amount of blood;
detecting the beam with the detector when the tissue is in a second blood state having a second amount of blood, wherein the tissue transitions from the first blood state to the second blood state by a pulse from a heartbeat of the subject;
obtaining an estimate of the second amount of blood; and
determining an estimate of a concentration of the analyte in the blood based on the output signal, the estimate of the first amount of blood, and the estimate of the second amount of blood.

14. The method of claim 13, wherein the analyte is glucose, and further comprising determining estimates of the concentration of the glucose in the blood for a predetermined number of heartbeats.

15. The method of claim 13, wherein, when the tissue transitions from the first blood state to the second blood state, a volume of blood increases in the tissue.

16. The method of claim 13, wherein the tissue transitions from the first blood state to the second blood state without exerting external pressure to the tissue.

17. A method for measuring an analyte in a fluid within a sample, comprising:
directing a radiation beam at the sample, the beam including at least two periods of radiation having different wavelengths, the analyte having different absorption coefficients for the two different wavelengths;
detecting the beam with a detector when the sample is in a first sample fluid state having a first amount of fluid, wherein the detector is configured to generate an output signal proportional to an intensity of the beam at each of the two different wavelengths;
obtaining an estimate of the first amount of fluid;
detecting the beam with the detector when the sample is in a second sample fluid state having a second amount of fluid, wherein the sample transitions from the first sample fluid state to the second sample fluid state by pulsations of the sample;
obtaining an estimate of the second amount of fluid; and
determining estimates of a concentration of the analyte in the fluid for a predetermined number of pulsations based on the output signals, the estimate of the first amount of fluid, and the estimate of the second amount of fluid.

18. The method of claim 17, wherein the pulsations are pulses from heartbeats.

19. The method of claim 17, wherein the second amount of fluid is larger than the first amount of fluid.

20. The method of claim 17, wherein the sample transitions from the first sample fluid state to the second sample fluid state without external pressure being applied to the sample.
